(19) United States
(12) Patent Application Publication
(10) Pub. No.: US 2022/0204964 A1
(43) Pub. Date: Jun. 30, 2022

(54) SYSTEMS AND METHODS FOR LIVING SILK ARTICLES

(71) Applicant: Trustees of Tufts College, Medford, MA (US)

(72) Inventors: David L. Kaplan, Concord, MA (US); Siwei Zhao, Medford, MA (US); Fiorenzo G. Omenetto, Lexington, MA (US)

(21) Appl. No.: 17/607,620

(22) PCT Filed: Apr. 29, 2020
(86) PCT No.: PCT/US2020/030570

§ 371 Date: Oct. 29, 2021

Related U.S. Application Data
(60) Provisional application No. 62/840,295, filed on Apr. 29, 2019.

(51) Int. Cl. C12N 11/04; C08J 3/24; C08J 3/075; C12P 3/00; B33Y 10/00; B33Y 70/00; B33Y 30/00; B33Y 80/00

(52) U.S. Cl. CPC C12N 11/04; C08J 3/24; C08J 3/075; C12P 3/00; C08J 2401/28; B33Y 70/00; B33Y 30/00; B33Y 80/00; C08J 2389/00; B33Y 10/00

(57) ABSTRACT

A method of making silk articles including preparing a silk fibroin solution including silk fibroin and microalgae, and introducing the silk fibroin solution into a solvent bath including a crosslinking agent. The method can incorporate 3D printing techniques to allow for easy fabrication of the articles into various forms. The silk articles can provide a cell-friendly matrix that allows 3D encapsulation of microalgae while maintaining normal cell proliferation and functions for an extended period of time.

### CROSS-REFERENCE TO RELATED APPLICATIONS

This application is based on, claims priority to, and incorporates herein by reference in its entirety, U.S. Provisional Patent Application Ser. No. 62/840,295, filed on Apr. 29, 2019 and entitled “System and Method for Living Silk Articles”.

### STATEMENT REGARDING FEDERALLY SPONSORED RESEARCH

This invention was made with government support under grant P41 EB002520 awarded by the National Institutes of Health. The government has certain rights in the invention.

### BACKGROUND

Carbon dioxide (CO₂) is one of the major greenhouse gas (GHG) emitted by human activities, accounting for more than 80% of the total GHG emissions in the U.S. CO₂ traps heat in the atmosphere by absorbing radiation within the infrared range. Excessive CO₂ emission contributes to the increasing surface temperature of Earth, which has detrimental impacts on environment, ecosystems and biodiversity. On a smaller scale, high level of carbon dioxide (>1,000 ppm) in residential or commercial buildings, often a result of poor ventilation and/or high human activity, can cause health issues, such as headaches and fatigue. Extremely high concentrations of carbon dioxide can cause nausea, dizziness, and vomiting.

Currently, carbon pollution is primarily mitigated by passive approaches, and the most effective way is the reduction of fossil fuel consumption. This requires changes in behavior, infrastructure upgrades, and significant technological development. More active methods have also been explored, including CO₂ capture and sequestration. However, these approaches are mainly suitable for large-scale power plants or industrial processes, due to the technological complexity and cost.

Various studies have estimated the CO₂ consumption from green algae ponds, which ranges from 0.07 to 0.21 ton per meter square per year. The CO₂ reduction rate is dependent on the surface area of the algal pond, based on how much light energy is absorbed. Algae have high growth rate due to their simple structures and high energy conversion efficiency. Industrial-scale algae production has been practiced for several decades at low cost. However, attempts at low-cost, stable encapsulation of algae in solid constructions have had limited success.

Silk has been used in prior systems to stabilize living animal cells in various applications in order to facilitate cell growth or stabilization. In such systems, the silk constructs act to temporarily stabilize the animal cells for eventual release from the construct. Applications for such constructs have included in vivo and in vitro tissue generation, where the animal cells are removed from the silk construct or the silk construct is degraded away in a subject.

Therefore, there is an unmet need for methods and compositions that allow for algae encapsulation in various commercial applications, in order to facilitate CO₂ reduction and oxygen generation.

### SUMMARY OF THE DISCLOSURE

The present disclosure address the aforementioned drawbacks by providing, in part, bio-compatible hydrogel materials that encapsulate photosynthetic organisms, such as green algae, to ensure viability and functionality. Silk fibroin was discovered to be a suitable base material for the hydrogel articles described herein, which provide a cell-friendly matrix that allows 3D encapsulation of microalgae while maintaining normal cell proliferation and functions. In some aspects, the unique combination of silk fibroin and hydroxypropyl methylcellulose (HPMC) allow for 3D printing and easy fabrication of the articles into various forms. Consequently, the methods, articles and systems of the present disclosure may provide traits that no existing GHG mitigation technologies can offer, including low cost, environmental compatibility, self-sustainability, and long-term functionality without human intervention.

In one aspect, the present disclosure provides a method of making a silk article. The method comprising preparing a silk fibroin solution comprising silk fibroin and microalgae; and introducing the silk fibroin solution into a solvent bath comprising a crosslinking agent.

In another aspect, the present disclosure provides a method of making a silk article. The method comprising preparing a silk fibroin solution comprising silk fibroin, horseradish peroxidase, and hydroxypropyl methylcellulose; and introducing the silk fibroin solution into a solvent bath comprising hydrogen peroxide.

In one aspect, the present disclosure provides a silk article comprising silk fibroin and microalgae, wherein the silk article is configured to allow the microalgae to undergo photosynthesis.

In another aspect, the present disclosure provides a silk article comprising silk fibroin, hydroxypropyl methylcellulose, and horseradish peroxidase.

In one aspect, the present disclosure provides a system for forming a silk article. The system comprising a source of silk fibroin solution, the silk fibroin solution comprising silk fibroin and microalgae; a solvent bath comprising a cross-linking agent; and an injector configured to introduce the silk fibroin solution into the solvent bath.

These and other advantages and features of the present invention will become more apparent from the following detailed description of the preferred aspects of the present invention when viewed in conjunction with the accompanying drawings. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention. It should be understood, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWINGS

The drawings described herein are for illustration purposes only and are not intended to limit the scope of the disclosed embodiments in any way. The drawings described herein are for illustrative purposes only of selected embodiments and not all possible implementations, and are not intended to limit the scope of any of the various embodiments. It is understood that the drawings are not drawn to scale.

FIG. 1 illustrates a flowchart of a method of making a silk article, in accordance with one aspect of the present disclosure.

FIG. 2 illustrates a flowchart of a method of making a silk article, in accordance with another aspect of the present disclosure.

FIG. 3 illustrates a silk article, in accordance with one aspect of the present disclosure. The illustration includes a magnified callout section.

FIG. 4 illustrates a system for forming a silk article, in accordance with one aspect of the present disclosure.

FIG. 5 illustrates dynamic viscosity of silk/HPMC solution mixtures at various silk to HPMC ratios for the experiment of Example 1.

FIG. 6 depicts gelation kinetics of various compositions for the experiment of Example 1. The four left panel plots illustrate the gelation kinetics of silk/HPMC hydrogel at various silk to HPMC ratios and HRP concentration. The right panel chart shows time to reach 80% gelation for each silk to HPMC ratio+/−SD.

FIG. 7 illustrates the Young's modulus of silk/HPMC hydrogel at various silk to HPMC ratios and HRP concentration after overnight crosslinking for the experiment of Example 1.

FIG. 8 illustrates the optical transmittance of silk/HPMC hydrogel at various silk to HPMC ratios and 180 unit/ml HPR concentration for the experiment of Example 1. The absorbance peaks for chlorophyll A (430 and 660 nm) and B (450 and 640 nm) were highlighted on the graph.

FIG. 9 illustrates the general printing process of silk/HPMC solution mixture containing microalgae for the experiment of Example 1.

FIG. 10A illustrates 3D printed structures (an Egyptian pyramid and a bar spanning two conical shaped pillars) using the solution mixture with a 6:4 silk to HPMC ratio and 180 unit/ml HRP for the experiment of Example 1. The insets showed the 3ds MAX designs. Bars showed 1 cm.

FIG. 10B illustrates a 3D printed microalgal “tree” consisting four layers (2 mm thick), along with an inset showing the 3ds MAX design for the experiment of Example 1. Bar show 1 cm.

FIG. 11 left panel illustrates phase contrast microscopic images of encapsulated microalgae at different time points showing long-term proliferation of microalgae within silk hydrogel matrix for the experiment of Example 1. Black arrows highlight dividing microalgal cells. Bar show 10 μm. Right panel shows average daily increase of cell number at different time points.

FIG. 12 illustrates dissolved oxygen concentration measured before and after each medium change over a 90-day period showing long-term photosynthetic activity of encapsulated microalgae for the experiment of Example 1.

FIG. 13A depicts a prophetic example of a residential home containing furniture and decorations which incorporate the articles of the present disclosure, in accordance with Example 2.

FIG. 13B depicts a prophetic example of a residential home containing windows which incorporate the articles of the present disclosure, in accordance with Example 2.

### DETAILED DESCRIPTION

Described herein are systems and methods that relate to algal containing structures, such as a silk hydrogel-algal containing structures. The systems and methods presented herein may be used for oxygen replenishment and carbon dioxide reduction in a variety of commercial applications. The present disclosure addresses the issues of carbon dioxide pollution by developing living architectures that allow sustainable, active removal of carbon dioxide from the environment using devices that can function for extended time frames. Living photosynthetic organisms (i.e. Microalgae) may be encapsulated in biocompatible silk hydrogels to ensure long-term viability and photosynthetic activity.

Among other applications, the articles described herein can be fabricated into arbitrary-shaped, multi-scale structures, while also functioning to remove carbon dioxide from the air. Such systems display traits that prior GHG mitigation technologies fail to offer, including low cost, environmental compatibility, self-sustainability, compostability and long-term functions without human intervention. The systems and method of the present disclosure can be utilized for small-scale carbon dioxide removal for indoor environments, or scaled up for industrial-scale carbon dioxide reduction. Microalgae has previously been used in food and various supplements. The articles described herein may be used as part of consumer products that are ingested or applied to the body.

The methods, articles, and systems of the present disclosure offer significant advantages over prior encapsulated algae constructions. For instance, the present disclosure can provide green, environmentally friendly technology because some aspects involve primarily naturally occurring materials (silk and microalgae) that are processed (printing, gelation) in a safe, aqueous, room temperature process. Moreover, silk hydrogel materials can be degraded by proteases, such as protease XIV and proteinase K, and the end products are low molecular weight peptides. This enables the articles of the present disclosure to be disintegrated and recycled for other uses, such as composting and generating biofuels after they complete their tasks. For indoor CO₂ reduction applications, safety is a top concern. In some aspects, the articles described herein do not involve any toxic chemicals. In such aspects, all materials may be both bio-compatible and edible.

The methods, articles, and systems of the present disclosure may incorporate 3D printing techniques to allow for easy fabrication of the articles into various forms. In some aspects, the unique combination of silk fibroin and hydroxypropyl methylcellulose allows for an ink solution with improved 3D printing results when compared to prior silk fibroin printing compositions. With regard to microalgae encapsulation in particular, the addition of hydroxypropyl methylcellulose as a thickening agent increases the viscosity of silk fibroin solution without compromising the cell-compatibility and bio-inertness of the resulting silk article. The increased viscosity allows for improved the 3D printing results, because the silk/HPMC ink is able to maintain its 3D shapes after being printed in aqueous solution. Gelatin, agar and alginate have been added in silk solution or other biological inks to achieve similar viscosity-increase effects. However, some of these materials require elevated temperature to melt (e.g. gelatin, agar), which may adversely affect the stability of silk fibroin solutions. Other materials are either difficult to dissolve in silk solution or are ionized in aqueous solution (e.g. alginate). The presence of high ion concentration in silk solution lowers the crosslinking efficiency. For the systems and methods described herein, HPMC presents significant advantages over the existing thickening agents, because 1) it dissolves at room temperature; 2) it mixes easily with silk solution; 3) it does not ionize in aqueous solution; and 4) it does not significantly interfere with the gelation of silk fibroin.

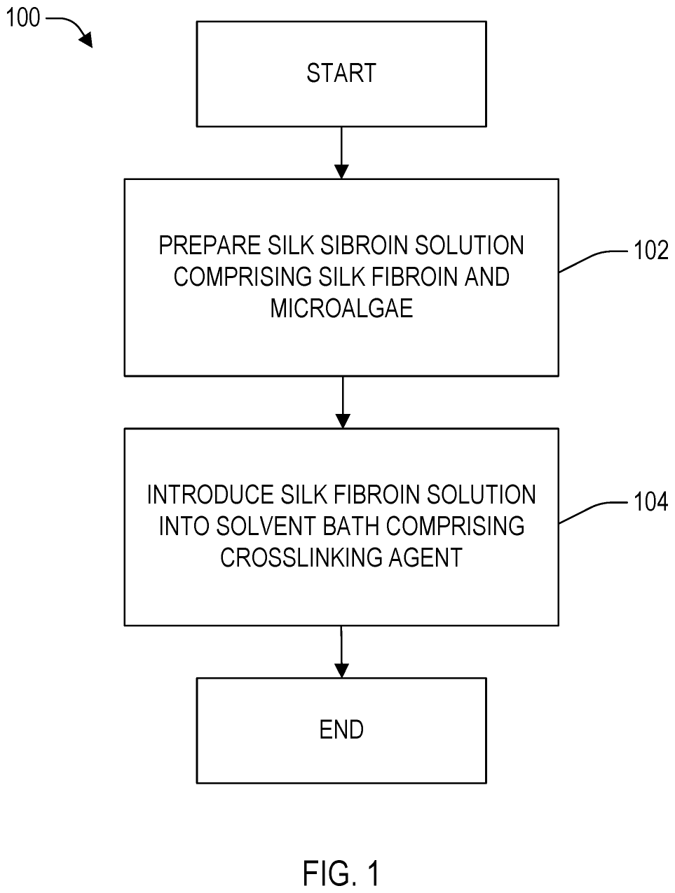

FIG. 1 depicts a method 100 of making a silk article. The method can include a first step 102 of preparing a silk fibroin solution comprising silk fibroin and microalgae, and a second step 104 of introducing the silk fibroin solution into a solvent bath comprising a crosslinking agent.

The first step 102 may include first forming a silk fibroin solution, which is then mixed with pre-cultivated microalgae. Alternatively or additionally, the first step may include loading a prepared silk fibroin solution into a device capable of enacting the second step. For instance, the first step may include loading an already prepared silk-fibroin solution into a 3D printing device. Likewise, preparing the silk fibroin solution may involve drawing an already prepared silk fibroin solution into a syringe or a similar device. Preparing the silk fibroin solution may involve applying one or more external stimuli or agents to an already prepared mixture of silk fibroin and microalgae. For instance, the first step 102 may include oxygenating a silk fibroin mixture over an extended period of time to induce microalgae proliferation prior to the second step.

As used herein, the term “silk fibroin” refers to silk fibroin protein whether produced by silkworm, spider, or other insect, or otherwise generated (Lucas et al., Adv. Protein Chem., 13: 107-242 (1958)). Any type of silk fibroin can be used in different embodiments described herein. Silk is naturally produced by various species, including, without limitation: Antheraea mylitta; Antheraea pernyi; Antheraea yamamai; Galleria mellonella; Bombyx mori; Bombyx mandarina; Galleria mellonella; Nephila clavipes; Nephila senegalensis; Gasteracantha mammosa; Argiope aurantia; Araneus diadematus; Latrodectus geometricus; Araneus bicentenarius; Tetragnatha versicolor; Araneus ventricosus; Dolomedes tenebrosus; Euagrus chisoseus; Plectreurys tristis; Argiope trifasciata; and Nephila madagascariensis. Silk fibroin produced by silkworms, such as Bombyx mori, is the most common and represents an earth-friendly, renewable resource. For instance, silk fibroin used in a silk film may be attained by extracting sericin from the cocoons of B. mori. Organic silkworm cocoons are also commercially available. There are many different silks, however, including spider silk (e.g., obtained from Nephila clavipes), transgenic silks, genetically engineered silks, such as silks from bacteria, yeast, mammalian cells, transgenic animals, or transgenic plants, and variants thereof, that can be used. See, e.g., WO 97/08315 and U.S. Pat. No. 5,245,012, each of which is incorporated herein as reference in its entirety.

Thus, in some embodiments, a silk solution is used to fabricate compositions of the present invention contain fibroin proteins, essentially free of sericins. In some embodiments, silk solutions used to fabricate various compositions of the present invention contain the heavy chain of fibroin, but are essentially free of other proteins. In other embodiments, silk solutions used to fabricate various compositions of the present invention contain both the heavy and light chains of fibroin, but are essentially free of other proteins.

Referring back to FIG. 1, the second step 104 may include injecting the silk fibroin solution into the solvent bath, wherein the injecting is adapted to form a predetermined three-dimensional shape. For instance, the prepared silk fibroin solution may be 3D printed into the solvent bath in a controlled manner. It should be recognized that numerous 3D printing systems and techniques can be applied to introduce the silk fibroin solution into the solvent bath. The silk fibroin solution may be iteratively layered to form a silk article having a predetermined structure. A desired structure may be specified to any 3D printing system in advance in order to tailor the form of the resulting silk article to the intended application.

Once introduced into the solvent bath, the silk fibroin solution may undergo hydrogelation in the solvent bath. To allow for this hydrogelation to occur, the method may include additional steps of incubating silk fibroin solution in the solvent bath for a period of time to undergo hydrogelation, and then removing the resulting silk article from the solvent bath. Once removed, the method may further comprise a step of placing the silk article in a medium that is suitable for microalgae proliferation. For instance, the resulting silk article may be removed and placed in a solution with a composition resembling seawater. Following introduction into the solvent bath and subsequent hydrogelation, the microalgae may be encapsulated within the resulting silk article.

The silk fibroin solution of the systems and methods of the present disclosure may comprise a chemical composition to support microalgae proliferation and sustained photosynthesis. The silk fibroin concentration in the silk fibroin solution may be from 10 wt % to 40 wt %, based on the total weight of the silk fibroin solution. In some embodiments, the silk fibroin concentration in the silk fibroin solution is at least 10 wt %, or at least 11 wt %, or at least 12 wt %, or at least 13 wt %, or at least 14 wt %, and at most 40 wt %, 30 wt %, 20 wt %, or about 15 wt %.

The microalgae may be a green algae. The microalgae may be of the Platymonas genus. In order to facilitate crosslinking and gelation upon contact with the solvent bath, the silk fibroin solution may comprise horseradish peroxidase. The concentration of horseradish peroxidase in the solution may be from 60 units/ml to 180 units/ml. In some embodiments, the horseradish peroxidase is present at a concentration of at least 60 units/ml, or at least 80 units/ml, or at least 100 units/ml, or at least 120 units/ml, or at most 140 units/ml, or at most 160 units/ml, or at most 180 units/ml. In some embodiments, the silk fibroin solution and the resulting silk article may comprise only nontoxic materials. The silk fibroin solution and the resulting silk article may be biodegradable. The silk fibroin solution and the resulting silk article may be safe for human consumption.

The silk fibroin solution may further comprise a thickening agent to allow the silk fibroin solution to have suitable properties to facilitate proper introduction into the solvent bath. The silk fibroin solution may have a thickening agent to support 3D printing of the solution into the solvent bath. The weight ratio between the silk fibroin to the thickening agent in the silk fibroin solution may range from 3:7 to 6:4, from 4:6 to 6:4, or specifically about 6:4. The concentration of the thickening agent in the silk fibroin solution may be from 10% w/v to 20% w/v, based on the total volume of the silk fibroin solution. The thickening agent may be include viscoelastic polymers including, but not limited to, hydroxypropyl methylcellulose.

The crosslinking agent within the solvent bath may be hydrogen peroxide. The total hydrogen peroxide concentration of the solvent bath may be at least 0.001% w/w, at least 0.05% w/w, at most 0.1% w/w, at most 0.05% w/w, or about 0.01% w/w. The solvent bath may have a chemical composition capable of supporting sustained proliferation of the microalgae. For instance, the solvent bath may comprise one or more salts, such as sodium chloride, in order to support marine microalgae strains. The solvent bath of the systems and methods of the present disclosure may specifically be absent of a photo-crosslinking agent or an organic solvent. For instance, the solvent bath may not comprise or may be substantially free of methanol, ethanol, isopropanol, butanol, ethylene glycol, propylene glycol, glycerol, and acetone.

Optical transparency and stiffness are important properties for evaluating the efficacy of any microalgae encapsulation article. The silk fibroin solution may be introduced into the chemical bath in a manner and have a chemical composition that produces a silk article that has a Young's modulus of at least 1 kPa, a Young's modulus of at least 10 kPa, a Young's modulus of at most 100 kPa, or a Young's modulus of at most 25 kPa. Likewise, the silk fibroin solution may be introduced into the chemical bath in a manner and have a chemical composition that produces a silk article that has a transmittance of at least 50%, at least 60%, or at least 70% over the visible range. Specifically, the optical transparency of the resulting silk article may have a transmittance of higher than 50%, 55%, 60%, 65%, 70%, or 80% at the absorbance peaks for chlorophyll A and B, the major chlorophylls in microalgae.

The resulting silk article of the method 100 may be capable of generating oxygen through photosynthesis of the microalgae. The silk article may be capable of generating oxygen through photosynthesis of the microalgae for at least 10, 30, 50, 70, 90, or 120 days. The resulting silk article may be characterized as supporting the survival of at least 50% of the total initial encapsulated microalgae population for at least 10, 30, 50, 70, 90, or 120 days.

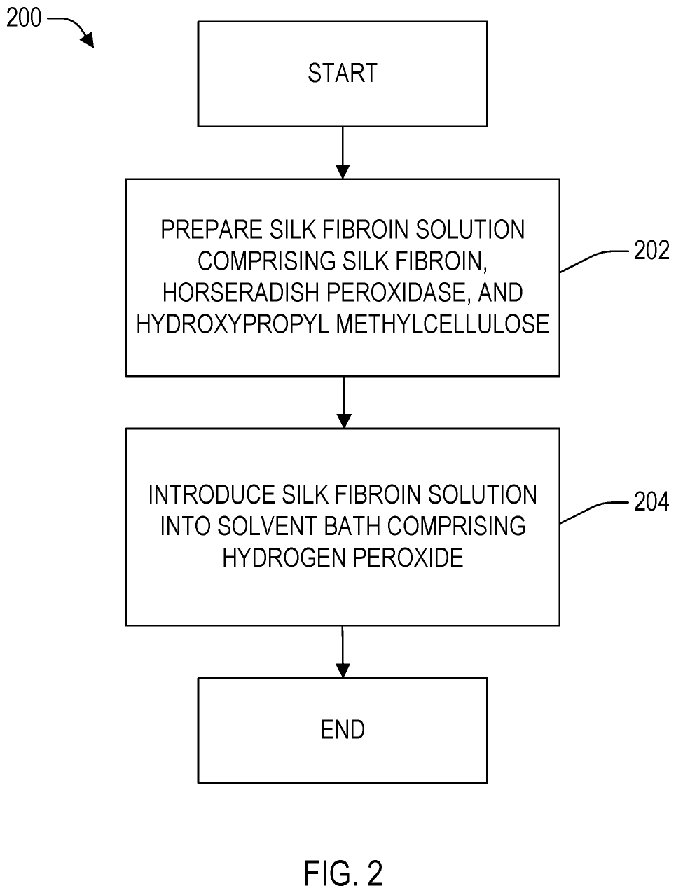

FIG. 2 depicts a method 200 of making a silk article. The method may comprise a first step 202 of preparing a silk fibroin solution comprising silk fibroin, horseradish peroxidase, and hydroxypropyl methylcellulose, and a second step 204 of introducing the silk fibroin solution into a solvent bath comprising hydrogen peroxide.

The first step 202 may include first forming a silk fibroin solution, which is then mixed with hydroxypropyl methylcellulose and horseradish peroxidase. Alternatively or additionally, the first step may include loading a prepared silk fibroin solution into a device capable of enacting the second step. For instance, the first step may include loading an already prepared silk-fibroin solution into a 3D printing device. Likewise, preparing the silk fibroin solution may involve drawing an already prepared silk fibroin solution into a syringe or a similar device. Preparing the silk fibroin solution may involve applying one or more external stimuli or agents to an already prepared mixture of silk fibroin and hydroxypropyl methylcellulose.

The second step 204 may include injecting the silk fibroin solution into the solvent bath in a manner that forms a predetermined three-dimensional shape. For instance, the prepared silk fibroin solution may be 3D printed into the solvent bath in a controlled manner. It should be recognized that numerous 3D printing systems and techniques can be applied to introduce the silk fibroin solution into the solvent bath. For instance, the silk fibroin solution may be iteratively layered to form a silk article having a predetermined structure. A desired structure may be specified to any 3D printing system in advance in order to tailor the form of the resulting silk article to the intended application.

Once introduced into the solvent bath through step 204, the silk fibroin solution may undergo hydrogelation in the solvent bath. To allow for this hydrogelation to occur, the method may include additional steps of incubating silk fibroin solution in the solvent bath for a period of time to undergo hydrogelation, and then removing the resulting silk article from the solvent bath.

The silk fibroin concentration in the silk fibroin solution of the method 200 may be from 10 wt % to 40 wt %, based on the total weight of the silk fibroin solution. In some embodiments, the silk fibroin concentration in the silk fibroin solution is at least 10 wt %, or at least 11 wt %, or at least 12 wt %, or at least 13 wt %, or at least 14 wt %, and at most 40 wt %, 30 wt %, 20 wt %, or about 15 wt %.

In some embodiments, the microalgae may be a green algae. The microalgae may be of the Platymonas genus. The concentration of horseradish peroxidase in the solution may be from 60 units/ml to 180 units/ml. In some embodiments, the horseradish peroxidase is present at a concentration of at least 60 units/ml, or at least 80 units/ml, or at least 100 units/ml, or at least 120 units/ml, or at most 140 units/ml, or at most 160 units/ml, or at most 180 units/ml. In some embodiments, the silk fibroin solution and the resulting silk article may comprise only nontoxic materials. The silk fibroin solution and the resulting silk article may be biodegradable. The silk fibroin solution and the resulting silk article may be safe for human consumption.

The weight ratio between the silk fibroin to hydroxypropyl methylcellulose in the silk fibroin solution may range from 3:7 to 6:4, from 4:6 to 6:4, or specifically about 6:4. The concentration of the hydroxypropyl methylcellulose in the silk fibroin solution may range from 10% w/v to 20% w/v.

The total hydrogen peroxide concentration of the solvent bath may be at least 0.001% w/w, at least 0.05% w/w, at most 0.1% w/w, at most 0.05% w/w, or about 0.01% w/w. The solvent bath of the systems and methods of the present disclosure may specifically be absent of a photo-crosslinking agent or an organic solvent.

In one aspect, the present disclosure provides a silk article formed by any of the methods described herein.

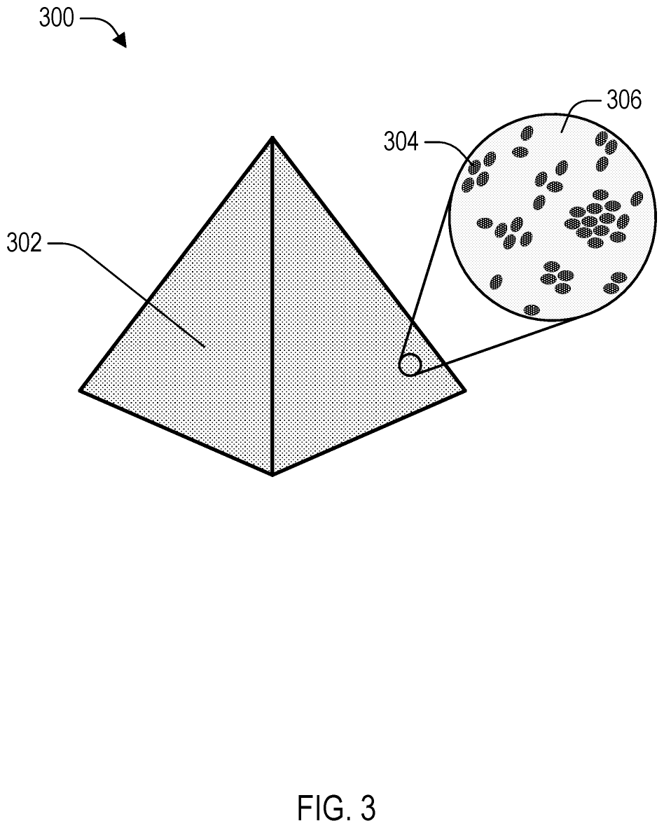

FIG. 3 depicts a silk article 300 comprising silk fibroin 304 and microalgae 306, wherein the silk article is configured to allow the microalgae to undergo photosynthesis. The depicted silk article 300 has a three-dimensional structure, such as a pyramid structure 302, but it should be appreciated that the silk article can take numerous forms depending on the intended application.

The silk article 300 may comprise a chemical composition to support microalgae proliferation and sustained photosynthesis. The silk fibroin concentration in the silk fibroin article may be from 10 wt % to 40 wt %, based on the total weight of the silk fibroin solution. In some embodiments, the silk fibroin concentration in the silk fibroin solution is at least 10 wt %, or at least 11 wt %, or at least 12 wt %, or at least 13 wt %, or at least 14 wt %, and at most 40 wt %, at most 30 wt %, or at most 20 wt %, or about 15 wt %.

In some embodiments, the microalgae may be a green algae. The microalgae may be of the Platymonas genus. The silk article 300 may comprise horseradish peroxidase. The concentration of horseradish peroxidase in the solution may be from 60 units/ml to 180 units/ml. In some embodiments, the horseradish peroxidase is present at a concentration of at least 60 units/ml, or at least 80 units/ml, or at least 100 units/ml, or at least 120 units/ml, or at most 140 units/ml, or at most 160 units/ml, or at most 180 units/ml. In some embodiments, the silk article may comprise only nontoxic materials. The silk article 300 may be biodegradable. The silk article 300 may be safe for human consumption.

The silk article 300 may further comprise a thickening agent. The weight ratio between the silk fibroin to the thickening agent in the silk fibroin solution may range from 3:7 to 6:4, from 4:6 to 6:4, or specifically about 6:4. The concentration of the thickening agent in the silk article 300 may be from 10% w/v to 20% w/v, based on the total volume of the silk fibroin solution. The thickening agent may be include viscoelastic polymers including, but not limited to, hydroxypropyl methylcellulose.

The silk article 300 may have a Young's modulus of at least 1 kPa, a Young's modulus of at least 10 kPa, of a Young's modulus of at most 100 kPa, or a Young's modulus of at most 25 kPa. The silk article 300 may have a transmittance of at least 50%, at least 60%, or at least 70% over the visible range. Specifically, the optical transparency of the silk article 300 may have a transmittance of higher than 50%, 55%, 60%, 65%, 70%, or 80% at the absorbance peaks for chlorophyll A and B, the major chlorophylls in microalgae.

The silk article 300 may be capable of generating oxygen through photosynthesis of the microalgae. The silk article 300 may be capable of generating oxygen through photosynthesis of the microalgae for at least 10, 30, 50, 70, 90, or 120 days. The silk article 300 may be characterized as supporting the survival of at least 50% of the total initial encapsulated microalgae population for at least 10, 30, 50, 70, 90, or 120 days.

In another aspect, the present disclosure provides a silk article comprising silk fibroin, hydroxypropyl methylcellulose, and horseradish peroxidase. The silk fibroin concentration in the silk fibroin article may be from 10% w/v to 40% w/v, from 10% w/v to 20% w/v, or specifically about 15% w/v. The concentration of horseradish peroxidase may be from 60 units/ml to 180 units/ml. The weight ratio between silk fibroin and hydroxypropyl methylcellulose in the silk fibroin article may be from 3:7 to 6:4, from 4:6 to 6:4, or specifically about 6:4. The concentration of the thickening agent in the silk article may be from 10% w/v to 20% w/v. The thickening agent may be hydroxypropyl methylcellulose.

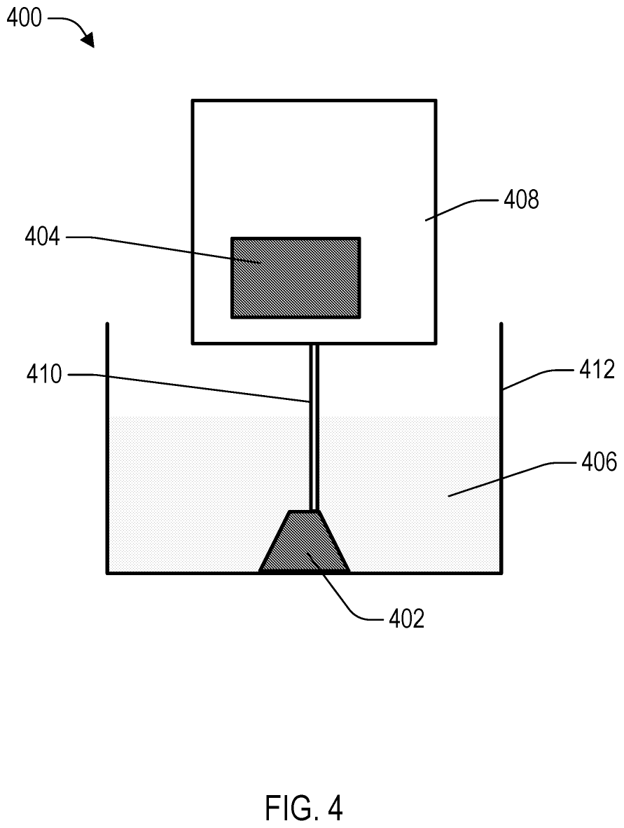

FIG. 4 depicts a system 400 for forming a silk article 402. The system may comprise a source of silk fibroin solution 404, the silk fibroin solution 404 comprising silk fibroin and microalgae. The system 400 may also include a solvent bath 406 comprising a cross-linking agent and contained within a solvent container 412. Additionally, the system 400 may include an injector 408 configured to introduce the silk fibroin solution 404 into the solvent bath 406 through an injector outlet 410. In some embodiments, the injector 408 includes a chamber for housing the silk fibroin solution 404 that is in fluid communication with the injector 410, which may be a nozzle or a needle.

The injector 408 may be configured to introduce the silk fibroin solution 404 into the solvent bath 406 in a manner that forms a predetermined three-dimensional shape. For instance, the injector 408 may be a 3D printer and the prepared silk fibroin solution 404 may be 3D printed into the solvent bath 406 in a controlled manner. It should be recognized that numerous 3D printing systems and techniques can be applied by the injector 408 to introduce the silk fibroin solution 404 into the solvent bath 406. For instance, the silk fibroin solution 404 may be iteratively layered to form a silk article 402 having a predetermined structure. A desired structure may be specified to the injector 408 in advance in order to tailor the form of the resulting silk article 402 to the intended application.

The solvent bath 406 may be configured to incubate the silk fibroin solution for a period of time to undergo hydrogelation and allow for the easy removal the resulting silk article 402 from the solvent bath 406. The silk fibroin solution 404 of the system 400 may comprise a chemical composition to support microalgae proliferation and sustained photosynthesis. The silk fibroin concentration in the silk fibroin solution 404 may be from 10 wt % to 40 wt %, based on the total weight of the silk fibroin solution. In some embodiments, the silk fibroin concentration in the silk fibroin solution is at least 10 wt %, or at least 11 wt %, or at least 12 wt %, or at least 13 wt %, or at least 14 wt %, and at most 40 wt %, at most 30 wt %, at most 20 wt %, or about 15 wt %.

In some embodiments, the microalgae may be a green algae. The microalgae may be of the Platymonas genus. In order to facilitate crosslinking and gelation upon contact with the solvent bath 406, the silk fibroin solution 404 may comprise horseradish peroxidase. The concentration of horseradish peroxidase in the solution may be from 60 units/ml to 180 units/ml. In some embodiments, the horseradish peroxidase is present at a concentration of at least 60 units/ml, or at least 80 units/ml, or at least 100 units/ml, or at least 120 units/ml, or at most 140 units/ml, or at most 160 units/ml, or at most 180 units/ml. The silk fibroin solution 404 may further comprise a thickening agent to allow the silk fibroin solution to have suitable properties to facilitate proper introduction into the solvent bath. The silk fibroin solution may have a thickening agent to support 3D printing of the solution by the injector 408. The weight ratio between the silk fibroin to the thickening agent in the silk fibroin solution may range from 3:7 to 6:4, from 4:6 to 6:4, or specifically about 6:4. The concentration of the thickening agent in the silk fibroin solution may be from 10% w/v to 20% w/v, based on the total volume of the silk fibroin solution. The thickening agent may be include viscoelastic polymers including, but not limited to, hydroxypropyl methylcellulose.

The crosslinking agent within the solvent bath 406 may be hydrogen peroxide. The total hydrogen peroxide concentration of the solvent bath may be between 0.001% w/w and 0.1% w/w, or specifically about 0.01% w/w. The solvent bath may have a chemical composition capable of supporting sustained proliferation of the microalgae. For instance, the solvent bath may comprise one or more salts, such as sodium chloride, in order to support marine microalgae strains. The solvent bath of the systems and methods of the present disclosure may specifically be absent of a photo-crosslinking agent or an organic solvent.

The injector outlet 410 may be a nozzle having a nozzle diameter of about 0.3, 0.4, 0.5, 0.6, or 0.7 mm. The injector may be configured to provide a constant pressure to the silk fibroin solution when introducing the silk fibroin solution into the solvent bath.

### EXAMPLES

The following examples set forth, in detail, ways in which articles containing microalgae and silk fibroin may be produced and utilized, and will enable one of skill in the art to more readily understand the principles thereof. The following examples are presented by way of illustration and are not meant to be limiting in any way.

### Example 1

A Inkredible™ 3D bioprinter (Cellink, Boston, Mass.) was used to print microalgae/silk solutions. The Inkredible™ bioprinter uses stable air pressure to extrude solution out of nozzle, which can allow for more constant solution flow and thus better consistency in printing quality. To prepare microalgae/silk solutions (i.e. the precursor of microalgal silk hydrogels), microalgae were harvested and concentrated at 1,200 rpm for 5 minutes. The concentrated microalgae solution was then mixed with the 26% w/v silk fibroin solution at 2:3 volume ratio, so the final silk concentration was 15.6% w/v. For solution mixtures that did not contain microalgae, only microalgal medium was added at this step. The microalgae/silk mixtures were then mixed with 15% w/v hydroxypropyl methylcellulose (HPMC) solution at various weight ratios (3:7, 4:6, 5:5 and 6:4, which are referred to as silk to HPMC ratio later) to increase the viscosity of the solution mixture to facilitate 3D printing. Finally, HRP was added to the solution mixture at different final concentrations (60, 120 and 180 unit/ml). The solution mixtures were then loaded in syringes that were specifically designed to work with the Inkredible™ bioprinters. The solution-loaded syringes were centrifuged at 2,000 rpm for 3 minutes to remove any air bubbles generated during the mixing of the solution. 3D models were designed with 3ds MAX (Autodesk, San Rafael, Calif., USA). The models were sliced and translated to G-code using Repetier-Host (Hot-World GmbH & Co. KG, Germany). A 0.5 mm layer height and 0.5 mm nozzle diameter were used for the printing. Blunt needles were used as printing nozzles. The air pressure for 3D printing was adjusted based on the viscosity of solution mixture to maintain proper solution flow so the thickness of each layer equaled the layer height (i.e., 0.5 mm). The solution was extruded into a medium (DI water for solution without microalgae, or microalgal medium for solution with microalgae) containing 0.01% w/w H₂O₂ to initiate crosslinking immediately after printing. This concentration of H₂O₂ was selected to allow efficient crosslinking to form the gels, while maintaining high cell survival. After the microalgal silk hydrogel was sufficiently crosslinked, the H₂O₂ containing medium was replaced with microalgal medium to support cell proliferation.

Materials

A marine microalgae strain, Platymonas sp. was used for the study. Platymonas is a microalgal strain that has been utilized in prior photosynthesis-related studies. The microalgae and its medium (Alga-Gro seawater medium) were obtained from Carolina Biological Supply Company (Burlington, N.C., USA). Raw silk cocoons produced by Bombyx mori silkworms were obtained from Tajima Shoji Co (Yokohama, Japan). Sodium carbonate, lithium bromide (LiBr), horseradish peroxidase (HRP), hydrogen peroxide (H₂O₂) and hydroxypropyl methylcellulose (HPMC) were purchased from Sigma-Aldrich (St. Louis, Mo., USA). Dialysis cassettes was purchased from Fisher Scientific (Pittsburgh, Pa., USA). 3D printing syringes were purchased from Nordson Medical (Marlborough, Mass., USA). Blunt needles were purchased from McMaster-Carr (Robbinsville, N.J., USA). Food coloring was obtained from a local grocery store.

Silk Processing

To prepare the silk fibroin solution, silk cocoons were diced into 1 cm² pieces. The cocoon pieces were then degummed by boiling in 0.2% w/w sodium carbonate solution for 30 min in order to remove the sericin. The degummed silk fibers were rinsed in copious amounts of water and allowed to dry for at least 12 hours. 9.3M LiBr solution was then used to dissolve silk fiber into fibroin solution at 60° C. for 4 hours. The silk fibroin solution was dialyzed against deionized (DI) water in dialysis cassettes with a 3,500 molecular weight cutoff for three days with at least 6 water changes to completely remove the LiBr content. Finally, the fibroin solution was centrifuged at 9,000 rpm at 4° C. for 20 min twice and filtered through a filter with 5 μm pore size to remove undissolved residues. The resulting solution contained only silk fibroin at 6% w/v and water. The silk fibroin solution was concentrated in dialysis cassettes in a fume hood to reach a concentration of 26% w/v, which was used later to prepare microalgae/silk mixtures for the hydrogel 3D printing.

Microalgae Culture

Microalgae were cultured according to the protocol recommended by the manufacturer. In brief, Platymonas sp. was cultured in suspension in sterile Erlenmeyer flasks at room temperature. Cool-white fluorescent lights at 2152 to 4304 lux were used for the initial 7 to 10 days to allow microalgae to grow. Light intensity was then lowered to 538 to 1076 lux to slow the growth for storage. A 16-hour light: 8-hour dark cycle was used. Constant air bubbling with air stone was used to supply oxygen to the medium to keep the oxygen concentration at 8.9 mg/L (100%). Microalgae concentration was calculated using a hemocytometer at the time of harvesting.

Characterization of 3D Printed Silk Hydrogel Materials

Ink mixtures at different silk to HPMC ratios were evaluated by rheometry (TA instruments) to characterize the dynamic viscosity. Solution mixtures were challenged with a range of shear rates from 0.001 s₋₁ to 1000 s⁻¹, and shear stress was used to calculate the viscosity. HRP with different final concentrations (60, 120 and 180 units/ml) was added to these solution mixtures, which were 3D printed in 0.01% H₂O₂ solution into a disc shape with 1.35 cm diameter and 2 mm height. The hydrogel discs were fully crosslinked overnight in H₂O₂ solution before they were tested for Young's modulus by Instron (model 3366, Norwood, Mass., USA). Mechanical compression and 40% maximum strain were used in these evaluations. Young's modulus was calculated at 20% strain. To characterize gelation kinetics of printed solutions, solution mixtures were 3D printed in black 96 well plate in H₂O₂ solution Immediately after printing, the intrinsic fluorescence of crosslinked phenolic groups at 415 nm was measured using a 315 nm excitation wavelength until a plateau was reached. The fraction of the maximum intensity, after subtracting background with H₂O₂, is reported as the degree of gelation. To measure the optical transparency of the printed structure, solution mixtures with 180 unit/ml HRP were printed into a disc shape with 2 cm diameter and 0.5 mm height in H₂O₂ solution and allowed to crosslink overnight. The optical absorbance was then measured from 350 nm to 750 nm, at 50 nm intervals, using a plate reader (SpectraMax M2, Molecular Devices). For all hydrogel materials characterization mentioned above, microalgae were not added.

Characterization of Microalgae Proliferation and Photosynthesis in 3D Printed Hydrogel Structures

All microalgal silk hydrogel structures were printed using a solution mixture with 6:4 silk to HPMC ratio and 180 unit/ml HRP concentration. This formula was selected due to optimal solution viscosity, gelation speed, mechanical stiffness and optical transparency. For microalgal proliferation evaluations, a 0.5 mm thick gel was printed in each well of a 24-well plate. The initial microalgal concentration was 7.5×10⁶ cells per milliliter of hydrogel. This low initial concentration was selected to allow room for microalgal proliferation and to facilitate easier cell counting. The microalgal silk hydrogels were then imaged using phase-contrast bright-field microscopy at days 0, 2, 6, 10, 17 and 30, and all cells that were in focus were counted to quantitatively calculate algal proliferation rate. The proliferation rate is presented as average daily increase of cell number (%),calculated by averaging the percent increase of cell numbers since the prior observation over the number of days since the prior observation. To characterize the photosynthesis of microalgal silk structures, microalgal hydrogel sheets of 5 cm wide, 10 cm long and 2 mm thick were printed and transferred to a BOD bottle. The initial microalgal concentration in these sheets was 5×10⁷ cells per milliliter of hydrogel. Then 30 ml microalgal medium was added in each bottle and the change in oxygen level was measured every 3 days using a needle-type optical oxygen sensor and a Microx 4 meter (both from PreSens, Germany). After each measurement, the medium was replaced. The oxygen concentration was reported as mg/L.

Statistics

Young's modulus of hydrogels, microalgal proliferation rate and oxygen production of microalgal silk structures were tested in duplicates and the data were presented as mean with standard deviation (+/−SD).

Results and Discussion

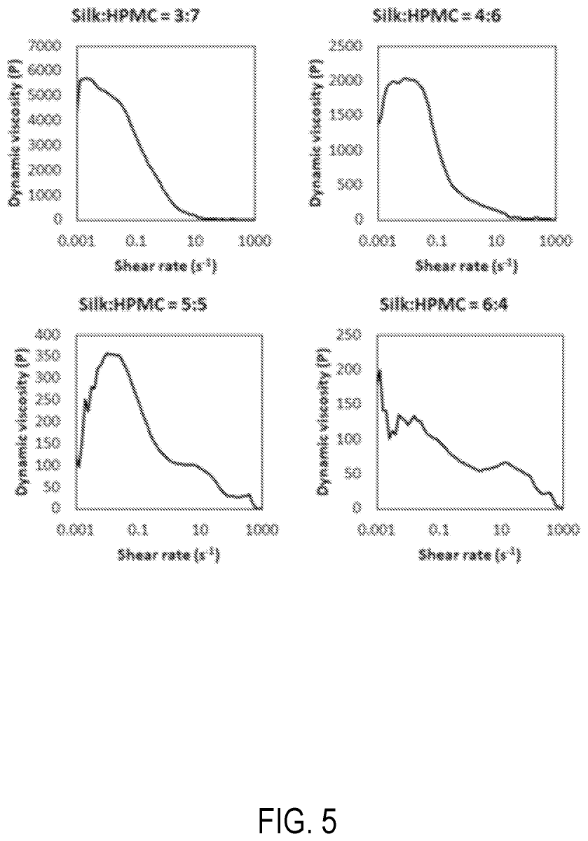

When choosing a thickening agent for silk hydrogel printing, the following criteria were considered: 1) room-temperature solubility; 2) easy dissolution in silk fibroin solution; and 3) absence of ionization in aqueous solution. Based on these criteria, hydroxypropyl methylcellulose (HPMC) was selected, which is chemically inert and biocompatible. We tested the dynamic viscosity of solution mixtures at several different silk to HPMC ratios (FIG. 5). All solution mixtures showed non-Newtonian behavior, which is typical for silk fibroin solution. At low silk to HPMC ratios (3:7, 4:6 and 5:5), the viscosity vs. shear rate curves first increased in the lower range of shear rate, indicating shear thickening behavior. The curves then reached a plateau and started to decrease with increasing shear rate, showing shear thinning. When the silk to HPMC ratio was increased to 6:4, the viscosity at lower shear rates was no longer stable, while the shear thinning behavior at higher shear rates was retained. The overall viscosity profile showed a linear relationship with HPMC content, where increasing HPMC content led to increased viscosity. These solution mixtures were later tested in 3D printing to determine which viscosity was sufficient to maintain the 3D shapes of printed filaments.

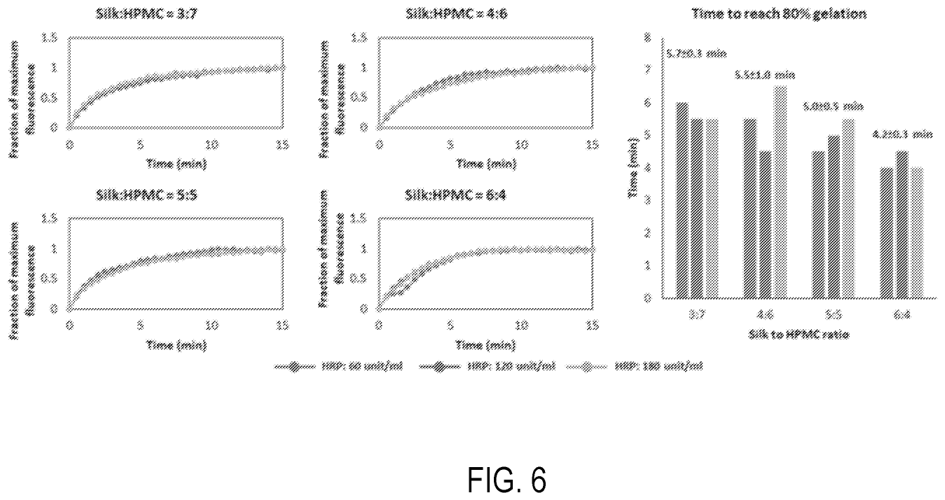

Next, we studied the gelation kinetics of the solution mixtures with different silk to HPMC ratios and HRP concentrations. It was suspected that there might be a positive correlation between silk fibroin gelation rate and HRP concentration, however the impact of HPMC content on the reaction kinetics was not clear. By examining the formation of phenolic crosslinking, with intrinsic fluorescence at 415 nm when excited at 315 nm, the gelation kinetics were quantified (FIG. 6). The gelation curves for each silk to HPMC ratio did not change significantly with changing HRP concentration, suggesting that the gelation rate was limited by the diffusion of H₂O₂, not the HRP concentration. Across different silk to HPMC ratios, solution mixtures with higher silk content tended to gel faster. The time to reach 80% gelation for solution mixtures with a 6:4 silk to HPMC ratio was 4.16±0.28 min, while solution mixtures with a 3:7 silk to HPMC ratio required 5.67±0.28 min to reach the same degree of gelation. These solution mixtures were tested by printing to determine which silk to HPMC ratio was most suitable to balance solution viscosity with gelation rate.

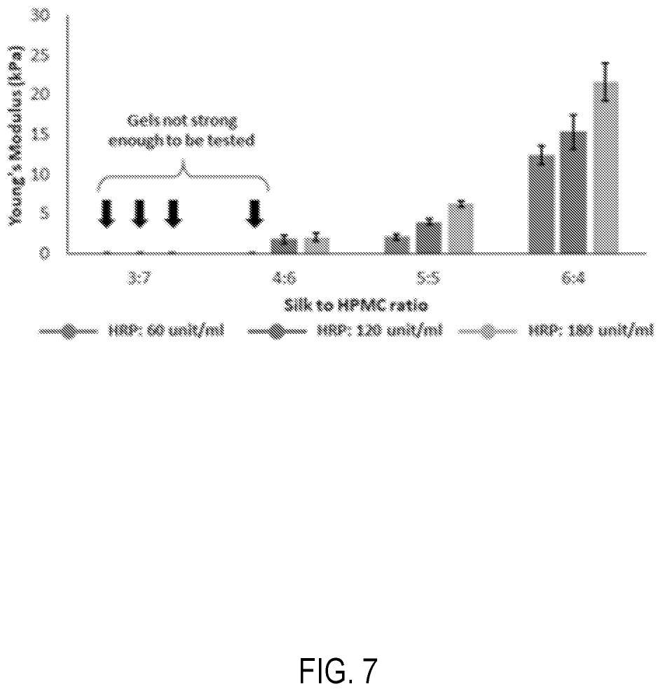

The addition of HPMC also influenced the Young's modulus of the hydrogel (FIG. 7). Hydrogels printed with solutions with higher silk contents tended to have higher mechanical strength. All solution mixtures with a 3:7 silk to HPMC ratio and solution mixtures with 4:6 silk to HPMC ratios and 60 unit/ml HRP did not form strong enough hydrogels to be tested, even after the overnight reactions, presumably due to low silk and HRP concentrations in the solution mixture. HRP concentration also had a positive impact on the final Young's modulus. Higher Young's modulus of final hydrogel was desired to enhance the mechanical stability of the printed structures. For the printing, we balanced solution viscosity with hydrogel mechanical strength to select the best solution formula for the 3D printing.

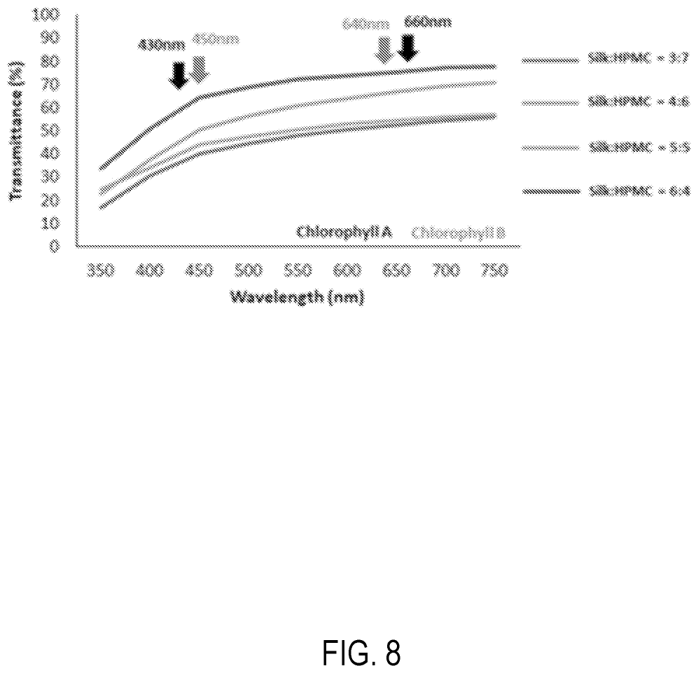

For microalgae immobilization, optical transparency is a key characteristic of the hydrogel material, as it directly affects light transmission and photosynthetic efficiency. Solution mixtures with different silk to HPMC ratios were printed in H₂O₂ solution and allowed to fully crosslink overnight. As can be seen in FIG. 8, the addition of HPMC had a significant impact on hydrogel optical transmission. Among the solution mixtures tested, the 6:4 silk to HPMC ratio provided the best optical transparency with higher than 60% transmittance at the absorbance peaks for chlorophyll A and B, the major chlorophylls in microalgae.

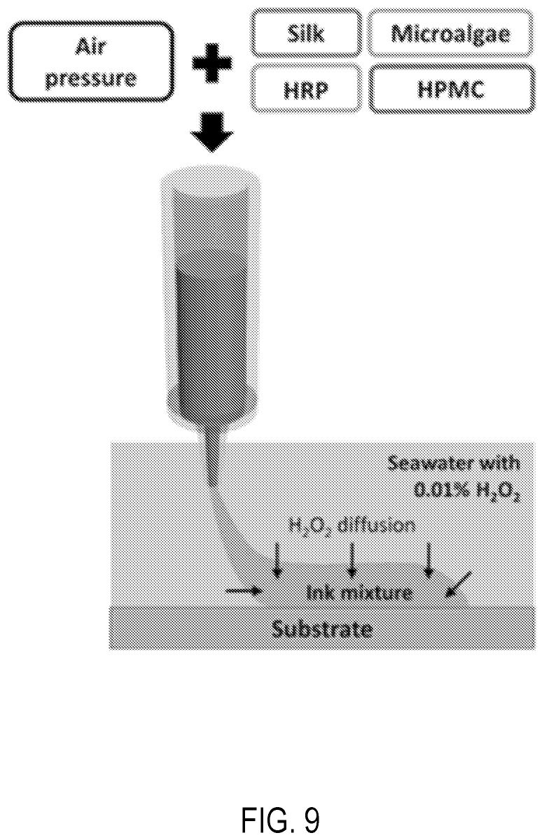

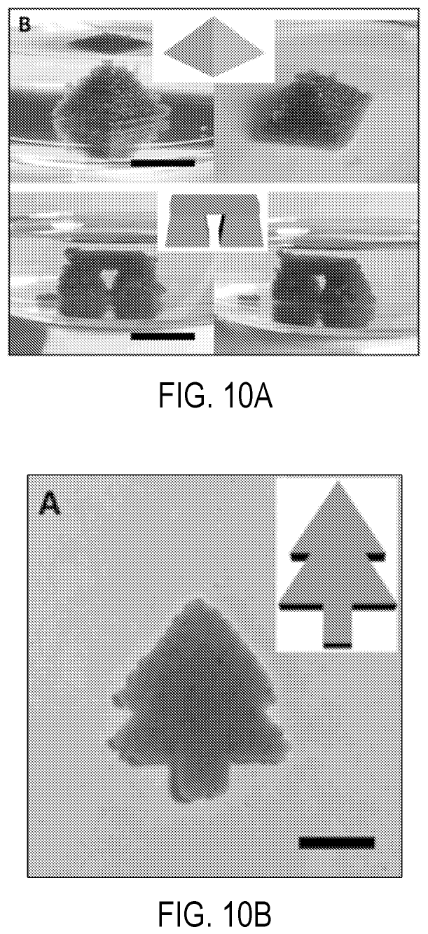

All solution mixtures formulated were tested for printability (FIG. 9). The solution mixture with a 6:4 silk to HPMC ratio and 180 unit/ml HRP generated printing outcomes that met the goals of the study: balancing viscosity, reaction kinetics, and mechanics. The viscosity was sufficiently high to allow the filament to maintain its 3D shape and a 0.5 mm layer height. The gelation rate was sufficiently rapid so the crosslinked hydrogel had the mechanical strength to maintain structural integrity. Premature gelation was not observed within the nozzle, thus it remained free of clogging during the entire printing process. 3D structures were successfully printed with the solution mixture with a 6:4 silk to HPMC ratio and 180 unit/ml HRP, such as an Egyptian pyramid and a bar spanning two conical shaped pillars to show the capacity of this silk/HPMC solution (FIG. 10A). Additional shapes were formed (FIG. 10B). The solution mixture used for the printing of these demonstrative 3D structures did not contain microalgae and was dyed with food coloring for enhanced contrast.

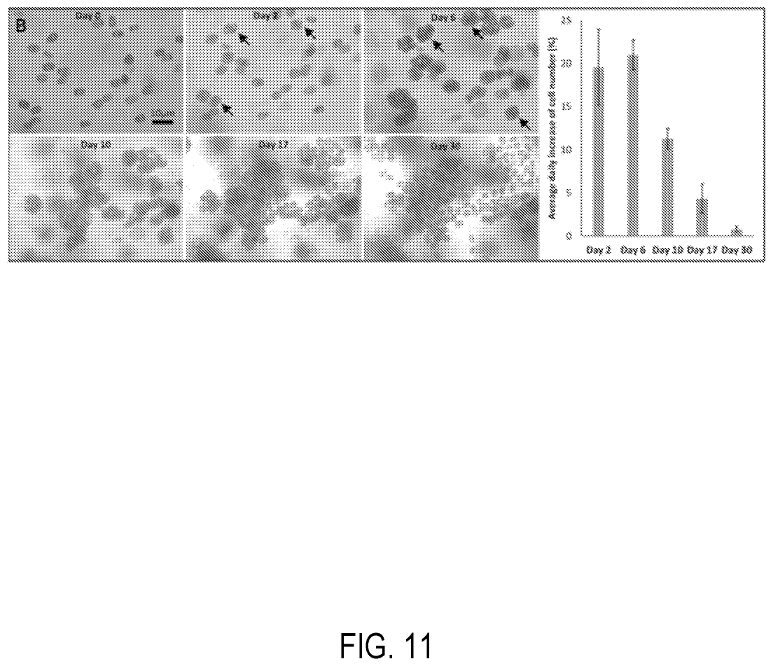

As a signature advantage of this new system, the silk hydrogel is able to provide a cell-friendly matrix that allows 3D encapsulation while maintaining normal cell proliferation and functions. Here, we wanted to determine whether the silk hydrogels could also support long-term 3D cultures of the microalgae and maintain photosynthetic activity. We added microalgae in the silk/HPMC solution mixture with a 6:4 silk to HPMC ratio right before printing. After the printed structures were completely crosslinked, the H₂O₂ containing medium was replaced with fresh microalgal medium to support the survival and proliferation of the microalgae. Microalgal proliferation was determined by counting cell number increases under phase contrast microscopy for four weeks post printing (FIG. 11). The encapsulated microalgae showed rapid proliferation at more than 10% per day for up to 10 days after printing, after which proliferation slowed and reached more steady state; consistent with general growth curves of the microalgae used in our study. The microalgae were not able to move within the hydrogel matrix due to the enzymatically crosslinked silk matrix.

However, the cells were able to divide and maintained their normal elliptical shape of 3 to 4 μm in length and 2 to 3 μm in width (FIG. 11). This may be considered advantageous for certain environmental applications to avoid release of the algae into the specific environment.

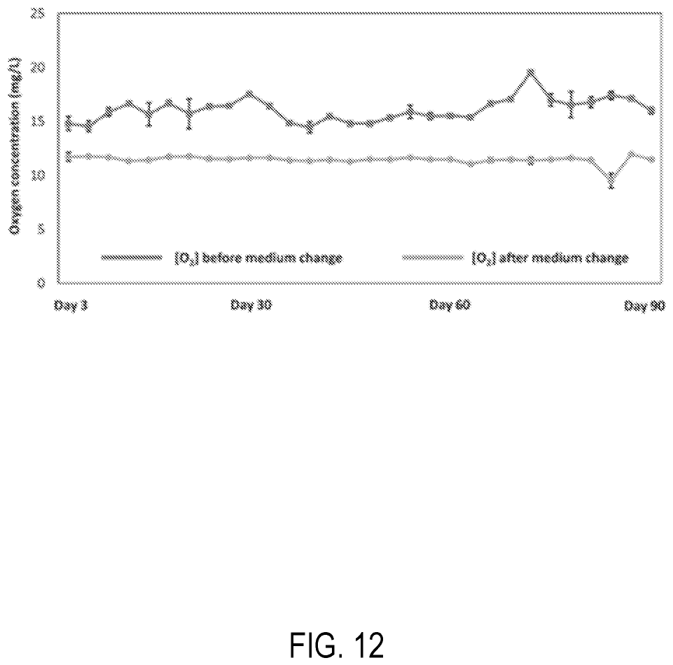

Using a non-invasive optical sensor, the photosynthetic activity of the 3D printed microalgal silk hydrogel structures was determined. The microalgal silk structures were immersed in microalgal medium, which was changed every three days and the oxygen concentration was measured before and after each medium change for 90 days. The test was conducted in BOD bottles with water seals to prevent gas exchange with the outside environment (FIG. 12). A consistent 3 to 8 mg/L oxygen concentration increase was observed for every three-day period, supporting the stability and long-term function of these microalgal silk hydrogel structures. This stability and long-term activity suggest that the system may be suitable for environmental applications such as indoor air improvements and CO₂ reduction with minimal human intervention.

One advantage of our system is that it is a green, environmentally friendly technology because it only involves naturally occurring materials (silk and microalgae) that are processed (printing, gelation) in a safe, aqueous, room temperature process. Moreover, silk hydrogel materials can be degraded by proteases, such as protease XIV and proteinase K, and the end products are low molecular weight peptides. This enables our systems to be disintegrated and recycled for other uses, such as composting and generating biofuels after they complete their tasks. For indoor applications, safety is a top concern. As aforementioned, our systems do not involve any toxic chemicals. All materials are both bio-compatible and even edible; there are numerous consumer products sold that are ingested or applied to the body. Silk has also been used to coat perishable food to extend shelf life, and is used in medical products as degradable systems. The consumption of microalgae as food and supplement also has a long history. Taken together, these unique advantages make our systems suitable for indoor and home utilities.

In conclusion, a microalgae/silk solution with mechanical properties and gelation kinetics useful for 3D printing was developed and successfully utilized to host microalgae in this experiment. The silk hydrogels provided a host environment to support the long-term proliferation and photosynthetic activity of encapsulated microalgae. Microalgae proliferation was demonstrated for more than 4 weeks and stable photosynthetic activity was observed for at least 90 days. The printability, stability and long-term functionality of such material supports potential environmental utilities.

### Example 2

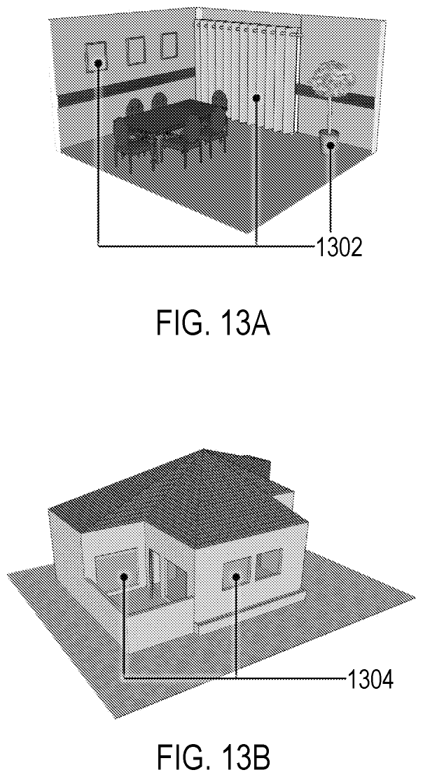

It is contemplated that incorporating the silk articles of the present disclosure into residential items capable of active removal of CO₂ from the environment. FIG. 13A depicts a residential home containing furniture and decorations 1302 which incorporate the microalgae articles of the present disclosure. FIG. 13B depicts a residential home containing windows 1304 which incorporate the microalgae articles of the present disclosure. We envision these living composite materials being fabricated into functional objects, while also functioning to remove carbon dioxide from the air. Such systems may display traits that prior GHG mitigation technologies struggle to offer, including low cost, environmental compatibility, self-sustainability, and long-term functionality without human intervention.

The present disclosure has been described one or more embodiments, and it should be appreciated that many equivalents, alternatives, variations, and modifications, aside from those expressly stated, are possible and within the scope of the invention.

1. A method of making a silk article, the method comprising:
A) preparing a silk fibroin solution comprising silk fibroin and microalgae; and
B) introducing the silk fibroin solution into a solvent bath comprising a crosslinking agent.

2. The method of claim 1, wherein the introducing of step B) comprises injecting the silk fibroin solution into the solvent bath, wherein the injecting is adapted to form a predetermined three-dimensional shape.

3. The method of claim 1 or 2, wherein the silk fibroin solution undergoes hydrogelation in the solvent bath.

4. The method of any one of the preceding claims, wherein the solvent bath does not include a photo-crosslinking agent or an organic solvent.

5. The method of any one of the preceding claims, wherein the solvent bath does not comprise an organic solvent selected from the group consisting of methanol, ethanol, isopropanol, butanol, ethylene glycol, propylene glycol, glycerol, and acetone.

6. The method of any one of the preceding claims further comprising:
C) incubating silk fibroin solution in the solvent bath to undergo hydrogelation; and
D) the removing the silk article from the solvent bath.

7. The method of any one of the preceding claims, wherein the silk fibroin concentration in the silk fibroin solution is at least 10 wt %, at least 12 wt %, or at least 14 wt %, and at most 40 wt %, 30 wt %, 20 wt %, or about 15 wt %.

8. The method of any one of the preceding claims, wherein the crosslinking agent is hydrogen peroxide.

9. The method of any one of the preceding claims, wherein the total hydrogen peroxide concentration of the solvent bath is at least 0.001% w/w, at least 0.05% w/w, at most 0.1% w/w, at most 0.05% w/w, or about 0.01% w/w.

10. The method of any one of the preceding claims, wherein the solvent bath comprises sodium chloride.

11. The method of any one of the preceding claims, wherein the solvent bath has a chemical composition capable of supporting sustained proliferation of the microalgae.

12. The method of any of the preceding claims, wherein the silk fibroin solution comprises horseradish peroxidase.

13. The method of any of the preceding claims, wherein the horseradish peroxidase is present at a concentration from 60 units/ml to 180 units/ml.

14. The method of any of the preceding claims, wherein the microalgae is encapsulated in the silk article following step B).

15. The method of any of the preceding claims, wherein the silk fibroin solution further comprises a thickening agent.

16. The method of any of the preceding claims, wherein the weight ratio between the silk fibroin to the thickening agent in the silk fibroin solution is from 4:6 to 6:4.

17. The method of any of the preceding claims, wherein the thickening agent is present in the silk fibroin solution at a concentration from 10% w/v to 20% w/v.

18. The method of any of the preceding claims, wherein the thickening agent is hydroxypropyl methylcellulose.

19. The method of any one of the preceding claims, wherein the silk fibroin solution is introduced into the chemical bath in a manner that produces a silk article that has a Young's modulus of at least 1 kPa, a Young's modulus of at least 10 kPa, of a Young's modulus of at most 100 kPa, or a Young's modulus of at most 25 kPa.

20. The method of any one of the preceding claims, wherein the silk fibroin solution is introduced into the chemical bath in a manner that produces a silk article that has a transmittance of at least 50%, at least 60%, or at least 70% over the visible range.

21. The method of any of the preceding claims, wherein the microalgae is a green algae.

22. The method of any of the preceding claims, wherein the microalgae is of the Platymonas genus.

23. The method of any of the preceding claims, wherein the silk article is capable of generating oxygen through photosynthesis of the microalgae.

24. The method of any of the preceding claims, wherein the silk article is capable of generating oxygen through photosynthesis of the microalgae for at least 90 days.

25. The method of any of the preceding claims, wherein the silk article is safe for human consumption.

26. A method of making a silk article, the method comprising:
A) preparing a silk fibroin solution comprising silk fibroin, horseradish peroxidase, and hydroxypropyl methylcellulose;
B) introducing the silk fibroin solution into a solvent bath comprising a hydrogen peroxide.

27. The method of claim 25, wherein the introducing of step B) comprises injecting the silk fibroin solution into the solvent bath, wherein the injecting is adapted to form a predetermined three-dimensional shape.

28. The method of claim 25 or 26, wherein the silk fibroin solution undergoes hydrogelation in the solvent bath.

29. The method of any one of the preceding claims, wherein the solvent bath does not include a photo-crosslinking agent or an organic solvent.

30. The method of any one of the preceding claims, wherein the solvent bath does not comprise a compound selected from the group consisting of methanol, ethanol, isopropanol, butanol, ethylene glycol, propylene glycol, glycerol, and acetone.

31. The method of any one of the preceding claims further comprising:
C) incubating silk fibroin solution in the solvent bath to undergo hydrogelation; and
D) the removing the silk article from the solvent bath.

32. The method of any of the preceding claims, wherein the silk fibroin concentration in the silk fibroin solution is at least 10 wt %, at least 12 wt %, or at least 14 wt %, and at most 40 wt %, 30 wt %, 20 wt %, or about 15 wt %.

33. The method of any of the preceding claims, wherein the total hydrogen peroxide concentration of the solvent bath is at least 0.001% w/w, at least 0.05% w/w, at most 0.1% w/w, at most 0.05% w/w, or about 0.01% w/w.

34. The method of any of the preceding claims, wherein the horseradish peroxidase is present at a concentration from 60 units/ml to 180 units/ml.

35. The method of any of the preceding claims, wherein the weight ratio between the silk fibroin and hydroxypropyl methylcellulose in the silk fibroin solution is from 4:6 to 6:4.

36. The method of any of the preceding claims, wherein the hydroxypropyl methylcellulose is present at a concentration from 10% w/v to 20% w/v.

37. The method of any of the preceding claims, wherein the silk article is safe for human consumption.

38. A silk article formed by the method of any one of the preceding claims.

39. A silk article comprising silk fibroin and microalgae, wherein the silk article is configured to allow the microalgae to undergo photosynthesis.

40. The silk fibroin article of claim 39, wherein the silk fibroin article is a hydrogel.

41. The silk article of claim 39 or 40, wherein the silk article further comprises horseradish peroxidase.

42. The silk article of any of the preceding claims, wherein the horseradish peroxidase is present at a concentration from 60 units/ml to 180 units/ml.

43. The silk article of any of the preceding claims, wherein the microalgae is encapsulated in the silk article.

44. The silk article of any of the preceding claims, further comprising hydroxypropyl methylcellulose.

45. The silk article of any of the preceding claims, wherein the weight ratio between the silk fibroin and hydroxypropyl methylcellulose in the silk article is from 4:6 to 6:4.

46. The silk article of any of the preceding claims, wherein the hydroxypropyl methylcellulose in the silk article at a concentration from 10% w/v to 20% w/v.

47. The silk article of any of the preceding claims, wherein the silk article has a Young's modulus of at least 1 kPa, a Young's modulus of at least 10 kPa, of a Young's modulus of at most 100 kPa, or a Young's modulus of at most 25 kPa.

48. The silk article of any of the preceding claims, wherein the silk article has a transmittance of at least 50%, at least 60%, or at least 70% over the visible range.

49. The silk article of any of the preceding claims, wherein the microalgae is a green algae.

50. The silk article of any of the preceding claims, wherein the microalgae is of the Platymonas genus.

51. The silk article of any of the preceding claims, wherein the silk article is capable of generating oxygen through photosynthesis of the microalgae for at least 90 days.

52. The silk article of any of the preceding claims, wherein the silk article is safe for human consumption.

53. A silk article comprising silk fibroin, hydroxypropyl methylcellulose, and horseradish peroxidase.

54. The silk fibroin article of claim 53, wherein the silk fibroin article is a hydrogel.

55. The silk fibroin article of claim 53 or 54, wherein the silk fibroin concentration in the silk article is at least 10 wt %, at least 12 wt %, or at least 14 wt %, and at most 40 wt %, 30 wt %, 20 wt %, or about 15 wt %.

56. The silk article of any of the preceding claims, wherein the horseradish peroxidase is present at a concentration from 60 units/ml to 180 units/ml.

57. The silk article of any of the preceding claims, wherein the weight ratio between the silk fibroin and hydroxypropyl methylcellulose in the silk article is from 4:6 to 6:4.

58. The silk article of any of the preceding claims, wherein the hydroxypropyl methylcellulose in the silk article is present at a concentration from 10% w/v to 20% w/v.

59. A system for forming a silk article, the system comprising:
a source of silk fibroin solution, the silk fibroin solution comprising silk fibroin and microalgae;
a solvent bath comprising a cross-linking agent; and
an injector configured to introduce the silk fibroin solution into the solvent bath.

60. The system of claim 59, wherein the injector is configured to introduce the silk fibroin solution into the solvent bath in a manner that forms a predetermined three-dimensional shape, thereby forming the silk article.

61. The system of any one of the preceding claims, wherein the solvent bath does not include a photo-crosslinking agent or an organic solvent.

62. The system of any one of the preceding claims, wherein the silk fibroin concentration in the silk fibroin solution is at least 10 wt %, at least 12 wt %, or at least 14 wt %, and at most 40 wt %, 30 wt %, 20 wt %, or about 15 wt %.

63. The system of any one of the preceding claims, wherein the crosslinking agent is hydrogen peroxide.

64. The system of any one of the preceding claims, wherein the total hydrogen peroxide concentration of the solvent bath is at least 0.001% w/w, at least 0.05% w/w, at most 0.1% w/w, at most 0.05% w/w, or about 0.01% w/w.

65. The system of any one of the preceding claims, wherein the source of silk fibroin solution comprises horseradish peroxidase.

66. The system of any one of the preceding claims, wherein the peroxidase is present at a concentration from 60 units/ml to 180 units/ml.

67. The system of any one of the preceding claims, wherein the source of silk fibroin solution further comprises a thickening agent.

68. The system of any one of the preceding claims, wherein the weight ratio between the source of silk fibroin and the thickening agent in the silk fibroin solution is from 4:6 to 6:4.

69. The system of any one of the preceding claims, wherein the thickening agent is present at a concentration from 10% w/v to 20% w/v.

70. The system of any one of the preceding claims, wherein the thickening agent is hydroxypropyl methylcellulose.
